(19) United States
(12) Patent Application Publication
(10) Pub. No.: US 2023/0190535 A1
(43) Pub. Date: Jun. 22, 2023

(54) METHOD OF MANUFACTURING WOUND TREATMENT MEDICAL TAPE

(71) Applicant: YUGENKAISHA CHOURYU, Shizuoka (JP)

(72) Inventor: Toshifumi HIRASHIMA, Shizuoka (JP)

(21) Appl. No.: 17/756,646

(22) PCT Filed: Mar. 17, 2022
(86) PCT No.: PCT/JP2022/012133

§ 371 Date: May 29, 2022

(30) Foreign Application Priority Data
Aug. 6, 2021 (JP) 2021-129983

(51) Int. Cl. A61F 13/02

(52) U.S. Cl. CPC A61F 13/0283

(57) ABSTRACT

There is provided a method of manufacturing a wound treatment medical tape capable of providing a high effect of preventing wound dehiscence by reducing a physiological tension acting on a wound and a skin around the wound in a dehiscence direction, and easy to use and apply regardless of whether the wound is sutured or not. The wound treatment medical tape has an elastic base member portion that covers a wound and a skin around the wound and has an elastic function, an adhesion portion having a function of applying and holding the elastic base member portion to the skin, a release portion having a function of protecting the adhesion portion, and an internal stress holding portion having a function of holding an effective shrink force in the elastic base member portion. The internal stress holding portion, the elastic base member portion, the adhesion portion, and the release portion are provided in this order from a bottom layer. The method includes: a step of applying a solution polymer for making the elastic base member portion to an upper layer of the internal stress holding portion; a step of subsequently holding the effective shrink force in the elastic base member portion with the internal stress holding portion by completing a film formation of the solution polymer with the effective shrink force being held in the elastic base member portion and concurrently completing a lamination of the elastic base member portion and the internal stress holding portion; and a step of subsequently laminating the adhesion portion and the release portion on a surface of the elastic base member portion opposite to the internal stress holding portion, and releases the effective shrink force existing in the elastic base member portion by removing the internal stress holding portion at a time of use.

### TECHNICAL FIELD

The present invention relates to a method of manufacturing a wound treatment medical tape for a physiological tension, and more particularly, to a method of manufacturing a wound treatment medical tape having an internal stress holding portion.

### BACKGROUND ART

A hurt formed by damaging part of a human body is called “injury”. The “injury” can be roughly classified into a “wound” which is an opened injury formed by cutting with a sharp knife or the like and “bruise” which is a closed injury formed by hitting with a blunt instrument or the like. Conventionally, in the field of the medical services, when a bottom of a wound which is an opened damage is shallow, treatment of closing and fixing the wound with an adhesive plaster, bandage, or the like is employed. When the wound bottom is deep, treatment of closing and fixing the wound with a suture thread or the like is widely employed.

In addition, the skin can be classified into three layers of the epidermis, the dermis, and the subcutaneous tissue from the upper layer. In general skin suturing, the three layers are sutured at once. Therefore, when a thick suture thread is used, a conspicuous scar is left on the skin surface in many cases. When a thin suture thread is used, wound dehiscence caused by an external force tends to increase. In order to prevent such a problem, “dermis suturing” has been selected and widely used as a suturing method. The dermis suturing is a suturing method in which the epidermis as a first layer of the skin is not sutured, but the dermis as a second layer and the subcutaneous tissue as a third layer are sutured. A suturing method in which only the epidermis as a first layer is sutured after dermis suturing is called “epidermis suturing”. In addition, in the dermis suturing, a thick suture thread (absorbent thread) that does not require thread removal is used, and a thin suture thread is selected for the epidermis suturing to minimize a scar on the skin surface.

However, even when the wound is closed and fixed through this treatment, the wound is re-opened often. Hereinafter, an event that “the wound that has been closed and fixed once is re-opened” will be referred to as “wound dehiscence”. One of the causes of the wound dehiscence is a “physiological and static sustained tension” (hereinafter, referred to as a “physiological tension”) caused by the skin and underlying muscles of the tissue surrounding the wound or the like, which acts on the tissue around the wound in the “direction of re-opening the wound” (hereinafter, referred to as a “dehiscence direction”).

Here, the wound dehiscence or the physiological tension will be described. Note that, in order to compare whether or not there is a physiological tension, “non-deteriorated tanned leather (animal hide)” is used as a sample not having the physiological tension. A non-deteriorated tanned leather having a length of 15 cm, a width of 10 cm, and a thickness of about 2 mm is prepared, and an incision having a straight line shape is made in the central part of the tanned leather with a surgical scalpel to a length of about 3 cm and a depth of about 1.5 mm. Then, the incision is sutured with a suture thread as in the case where a “skin of a living body” has an equivalent incision wound. In this case, when the suture thread is pulled with a strong force, the suture thread is broken, or the tanned leather is torn. This is also similarly applied to a case where the “skin of a living body” has an equivalent incision. When the suture thread is pulled with a strong force, the suture thread is broken, or the skin of a living body is torn. This is due to an immature stitching technique in the case of tanned leather, and an immature suturing technique in the case of skin. This problem is addressed by mastering a correct technique.

Even when the incised part of the tanned leather is stitched and closed on the basis of an appropriate stitching technique, the suture thread is broken, or the tanned leather is torn by pulling the tanned leather with a strong force perpendicularly to the close direction. Similarly, even when the incised wound of the skin is sutured with an appropriate suturing technique to close the wound, the suture thread is broken, or the skin of a living body is torn by applying a strong force to the wound in the dehiscence direction. When this happens unintentionally, the cause is an accident caused by an external force, and this is addressed by a preventive measure. This is a common phenomenon between the non-deteriorated tanned leather and the skin of a living body.

Here, the physiological tension will be visualized with reference to FIGS. 5 to 7. FIG. 5 is a schematic plan view illustrating a state of the wound surface after the skin is incised in a straight line shape. FIG. 6 illustrates the skin around the wound of FIG. 5 when it is sutured with a suture thread. FIG. 7 illustrates a force applied to a peripheral portion (wound edge) of the sutured wound of FIG. 6. Note that the black arrows in FIGS. 5 and 7 refer to the physiological tension 8, and the white arrows in FIG. 7 refers to a force 12 of attracting the wound surface by the suture thread. In addition, reference numeral 7 refers to the wound surface, reference numeral 9 refers to the suture thread, reference numeral 10 refers to the sutured wound surface, and reference numeral 11 refers to a portion through which the suture thread penetrates.

Next, the shape of the incision will be compared. In the case of tanned leather, the part incised with a surgical scalpel in a straight line shape is broken, but the shape of the incision remains linear in a straight line shape. In contrast, when the skin of a living body is incised in a straight line shape with a surgical scalpel, the wound shape is not limited to the straight line shape, and becomes an oval open shape as shown in the wound surface 7 of FIG. 5. This is due to the physiological tension 8 acting in the dehiscence direction indicated by the black arrows in FIG. 5. In addition, at the time of stitching and suturing, in the case of tanned leather having no physiological tension, the portion incised in a straight line shape may be stitched by aligning the cut ends. However, when the incised skin of a living body is sutured, it is necessary to pull the skin around the wound with a suture thread or practitioner’s fingers such that the shape of the wound surface (wound opening) to be sutured is changed from an oval shape to the straight line shape. This is because of the physiological tension acting in the dehiscence direction (physiological tension 8 in FIG. 5). In addition, the suture thread and the skin around the sutured wound are attractingly combined with each other after suturing by virtue of the force 12 of attracting the wound surface with the suture thread of FIG. 7 and the physiological tension 8 acting in the dehiscence direction.

In addition, comparing a thread spacing or a tightening state between the stitching of tanned leather and the suturing of the skin of a living body, in the case of the suturing of skin of a living body, the coarse spacing of the sutured thread of the skin around wound produces a better prognosis than the dense spacing. Therefore, the spacing becomes inevitably coarser, and the tightness of the suture thread is loosened to prevent overtightening. For this reason, when a wound that has been successfully sutured undergoes dehiscence several days later, the patient may easily attribute the cause to the doctor’s immature suturing technique, and the doctor may easily attribute it to the patient’s carelessness. Therefore, they do not think that the cause is the physiological tension, and a trouble often occurs between the patient and the doctor. Accordingly, presence of the physiological tension in the suturing causes the doctor’s annoyance. Note that, for wound dehiscence caused by necrosis of the skin or the like around the sutured wound due to poor circulation of blood or the like (medical tool compression wound caused by a suture thread), no effect is expected even by performing suturing again, and it is necessary to switch to conservative treatment in which all suture threads are removed, necrotic tissues are excised, the wound is covered with a covering to expect autotherapy in many cases. This is caused by the physiological tension acting on the skin around the wound, and is addressed by preventing the physiological tension acting on the skin around the wound. However, it is not easy to eliminate or reduce the physiological tension acting on the skin around the wound. Therefore, it was difficult to prevent wound dehiscence caused by the physiological tension acting in the dehiscence direction.

In some documents, a dehiscence prevention assistance tool having a function of consistently reducing the physiological tension in the dehiscence direction received by the tissue around the sutured wound and the tension acting on the suture thread associated with the physiological tension has been proposed (for example, see Patent Document 1). In this technique, a dehiscence prevention portion formed of elastic bare woven fabric (89% cotton and 11% polyurethane) is applied to the forearm by a holding portion having a function of holding the dehiscence prevention portion in close contact with the wound and the tissue around the wound by virtue of friction generated between the sutured part and the skin around the sutured part in order to reduce the physiological tension acting in the dehiscence direction. However, in the example of the dehiscence prevention assistance tool described in Patent Document 1, a twill weave fabric using cotton threads having no elastic function is thinned and is used as a string. Therefore, when the string is too strongly tightened, it will cause a pain to the patient. When the string is too weakly tightened, it is difficult to efficiently reduce the physiological tension acting in the dehiscence direction. Accordingly, it is not easy to efficiently reduce the physiological tension with a conventional dehiscence prevention assistance tool, and there has been no means for appropriately addressing such a disadvantage in the current situation.

### [Citation List]

### [Patent Document]

Patent Document 1: Japanese Patent No. 4790091

### SUMMARY OF INVENTION

In view of the aforementioned problems, it is therefore an object of the present invention to provide a method of manufacturing a wound treatment medical tape capable of providing a high effect of preventing wound dehiscence by reducing a physiological tension acting on a wound and skin around the wound in a dehiscence direction, and easy to use and apply regardless of whether the wound is sutured or not.

In order to address the aforementioned problems, the inventor made diligent studies over and over and completed the present invention as a result. Specifically, a wound treatment medical tape obtained through the manufacturing method according to the present invention has an elastic base member portion that covers a wound and a skin around the wound and has an elastic function, an adhesion portion having a function of applying and holding the elastic base member portion to the skin, a release portion having a function of protecting the adhesion portion, and an internal stress holding portion having a function of holding an effective shrink force in the elastic base member portion. The internal stress holding portion, the elastic base member portion, the adhesion portion, and the release portion are provided in this order from a bottom layer. In addition, a method of manufacturing the medical tape obtained through the manufacturing method according to the present invention includes, in a manufacturing process of the medical tape, a step of applying a solution polymer for making the elastic base member portion to an upper layer of the internal stress holding portion; a step of subsequently holding the effective shrink force in the elastic base member portion with the internal stress holding portion by completing a film formation of the solution polymer with the effective shrink force being held in the elastic base member portion and concurrently completing a lamination of the elastic base member portion and the internal stress holding portion; and a step of subsequently laminating the adhesion portion and the release portion on a surface of the elastic base member portion opposite to the internal stress holding portion. In addition, the method releases the effective shrink force existing in the elastic base member portion by removing the internal stress holding portion at a time of use. In the medical tape manufacturing method according to the present invention, any step other than those described above may be arbitrarily added without a particular limitation as long as the effects of the present invention can be obtained. Note that the effective shrink force refers to a shrink force, which can be obtained through the manufacturing process according to the present invention, is held in the elastic base member portion by a function of the internal stress holding portion, and can be released by removing the internal stress holding portion at the time of use, so that the elastic base member portion is shrunken by the released shrink force in a direction resisting the physiological tension to reduce the physiological tension.

In the wound treatment medical tape obtained through the manufacturing method according to the present invention, a means for consistently reducing the physiological tension acting on the sutured wound and the skin around the wound in the dehiscence direction is incorporated as a function of the wound treatment medical tape, and particularly, its shape is a tape. As a result, it is possible to apply the medical tape by covering the sutured wound and the skin around the wound, efficiently reduce the physiological tension, and provide a function of consistently reducing the physiological tension acting on the sutured wound and the skin around the wound in the dehiscence direction. In addition, in the medical tape manufacturing process, a function of holding the effective shrink force in the elastic base member portion is provided. At the time of use, the medical tape can be applied to the skin with the effective shrink force being held in the elastic base member portion. Furthermore, by removing the internal stress holding portion after applying the adhesion portion to the skin, the effective shrink force held in the elastic base member portion is released to the wound and the skin around the wound to which the adhesion portion is applied. The released effective shrink force efficiently acts in the direction resisting the physiological tension applied in the dehiscence direction, so that the elastic base member portion is shrunken in the direction resisting the physiological tension, and the wound and the skin around the wound are shrunken in the direction resisting the physiological tension applied in the dehiscence direction. As a result, it is possible to slightly loosen the skin around the sutured wound from the direction resisting the dehiscence direction to the sutured wound and consistently reduce the physiological tension acting on the skin around the sutured wound in the dehiscence direction and the tension acting on the suture thread in association with the physiological tension. Furthermore, since the medical tape has a function of reducing the physiological tension on the entire surface, and can be applied by covering the skin as a surface, the released effective shrink force acts not only on the wound and the skin around the wound covered with the medical tape, but also on the skin around a medical tape application portion, so as to generate a force of attracting the skin around the wound caused by the effective shrink force. As a result, it is possible to stably retain the wound and its surrounding tissue, consistently reduce the physiological tension, prevent wound dehiscence, and remove necessity of suturing depending on the wound condition in some cases. In addition, it is possible to prevent dehiscence of a wound that is generally difficult to suture, such as a contusion or bite wound. Furthermore, it is possible to stably retain the wound and its surrounding tissues and accelerate autotherapy regardless of whether the wound is sutured or not. According to the medical tape manufacturing method according to the present invention, it is possible to efficiently manufacture the medical tape.

### BRIEF DESCRIPTION OF DRAWINGS

FIG. 1 is a diagram illustrating a medical tape manufacturing process according to a first embodiment of the present invention, and is a schematic cross-sectional view illustrating a laminating process in which a solution polymer 1 for making an elastic base member portion 2 is applied to an upper layer of an internal stress holding portion 5.

FIG. 2 is a diagram illustrating a medical tape manufacturing process according to the first embodiment of the present invention, and is a schematic cross-sectional view illustrating a process of completing a film formation of the solution polymer 1 with the effective shrink force being provided in the elastic base member portion 2, concurrently completing a lamination of the elastic base member portion 2 and the internal stress holding portion 5, and holding the effective shrink force in the elastic base member portion 2 by the internal stress holding portion 5.

FIG. 3 is a diagram illustrating a medical tape manufacturing process according to the first embodiment of the present invention, and is a schematic cross-sectional view illustrating a process of laminating an adhesion portion 3 and a release portion 4 on a surface of the elastic base member portion 2 opposite to the internal stress holding portion 5.

FIG. 4 is a diagram illustrating a medical tape manufacturing process according to a second embodiment of the present invention, and is a schematic cross-sectional view illustrating an internal stress holding portion 6 formed in a substantially arcuate shape and used as the internal stress holding portion.

FIG. 5 is a schematic plan view illustrating a state of the wound surface after the skin is incised in a straight line shape.

FIG. 6 is a schematic plan view illustrating a state of the skin around the wound sutured with a suture thread of FIG. 5.

FIG. 7 is a schematic plan view illustrating a force applied to a peripheral portion (wound edge) of the sutured wound of FIG. 6.

FIG. 8 is a schematic plan view illustrating a medical tape according to the second embodiment of the present invention.

FIG. 9 is a schematic plan view illustrating a medical tape according to the second embodiment of the present invention used for the sutured wound of FIG. 6.

FIG. 10 is a schematic cross-sectional view illustrating the effects of using the medical tape according to the second embodiment of the present invention.

### DESCRIPTION OF EMBODIMENTS

In the process of diligent studies, the inventor found that it is important to consistently reduce the physiological tension acting on a skin around the sutured wound or the suture thread in the dehiscence direction in order to prevent dehiscence of the sutured wound. In addition, it is also necessary to stably hold the skin around the sutured wound in order to accelerate autotherapy after suturing. However, it is difficult to stably hold the suture thread because the skin around the sutured wound is consistently subjected to the physiological tension in the dehiscence direction.

Here, consistently reducing the physiological tension acting on the skin around the sutured wound in the dehiscence direction and the tension acting on the suture thread associated with the physiological tension by slightly loosening the skin around the sutured wound for the sutured wound from the direction resisting the dehiscence direction becomes an effective means for preventing the wound dehiscence caused by suture thread cutting subsequent to the suturing and the wound dehiscence caused by necrosis or the like of the skin around the sutured wound by the suture thread. By incorporating this effective means into a function of the wound treatment medical tape in the form of a surface covering to the skin, it is possible to provide a function of reducing the physiological tension on the entire surface and apply the tape by covering the sutured wound and the skin around the wound as a surface. Therefore, it is possible to consistently reduce the physiological tension acting on the skin around the sutured wound in the dehiscence direction and the tension acting on the suture thread associated with the physiological tension and prevent wound dehiscence caused by suture thread cutting subsequent to suturing and wound dehiscence caused by necrosis or the like of the skin around the sutured wound by the suture thread. In addition, since it is possible to consistently reduce the physiological tension acting on the dehiscence direction, it is possible to eliminate necessity of the suturing depending on the wound condition and expect dehiscence prevention for a wound generally difficult to suture, such as a contusion or bite wound. Furthermore, it is possible to stably hold the skin around the wound regardless of whether the wound is sutured or not and accelerate autotherapy. From this point of view, it is recognized that, in the conventional dehiscence prevention assistance tool, the physiological tension acting on the tissue around the sutured wound or the physiological tension acting on the suture thread in the dehiscence direction has not been considered.

In this regard, in order to provide a wound treatment medical tape formed by incorporating a function of consistently reducing the physiological tension acting on the skin around the sutured wound in the dehiscence direction and the tension acting on the suture thread associated with the physiological tension into the manufacturing process, the inventor selected materials of this medical tape as follows. That is, the inventor selected, as a material of the elastic base member portion that covers the wound and the skin around the wound and has an elastic function, a solution polymer material that is usable for a medical purpose and capable of providing an elastic function after a film formation and having an effective shrink force generated by an internal stress associated with forming shrinkage, resisting the physiological tension acting on the skin around the sutured wound in the dehiscence direction. In addition, as a material of the internal stress holding portion, an inelastic material to an upper layer of which the solution polymer for making the elastic base portion was applied in the manufacturing procedure and which had a function of holding the effective shrink force in the elastic base portion was selected. Then, as a material of the adhesion portion, a medical adhesive that has a function of applying and holding the elastic base member portion to the skin and has less adhesive residue and keratin damage at the time of releasing was selected. In addition, as the material of the release portion, a release sheet having a function of protecting the adhesion portion was selected. The inventor proposed an idea for a method of manufacturing the wound treatment medical tape by using functions having each material.

In the manufacturing process for the wound treatment medical tape by combining these materials, the wound treatment medical tape is obtained through a step of applying a solution polymer for making the elastic base member portion to the upper layer of the internal stress holding portion, a step of subsequently completing the film formation of the solution polymer with the effective shrink force being provided in the elastic base member portion, and completing a lamination of the elastic base member portion and the internal stress holding portion to hold the effective shrink force in the elastic base member portion with the internal stress holding portion, and a step of subsequently laminating the adhesion portion and the release portion on a surface of the elastic base member portion opposite to the internal stress holding portion.

The medical tape obtained through the manufacturing method according to the present invention has the internal stress holding portion, the elastic base member portion, the adhesion portion, and the release portion sequentially from the bottom. At the time of use of the wound treatment medical tape according to the present invention, the release portion is removed, the wound and the skin around the wound are covered with the elastic base member portion, and the adhesion portion is applied to the skin. Then, the internal stress holding portion is removed. Since the internal stress holding portion has a role of holding the effective shrink force existing in the elastic base member portion inside the elastic base member portion, the effective shrink force held in the elastic base member portion is released to the wound and the skin around the wound to which the adhesion portion is applied by removing the internal stress holding portion. In this case, the released effective shrink force efficiently acts in the direction resisting the physiological tension applied in the dehiscence direction to shrink the elastic base member portion in the direction resisting the physiological tension and shrink the wound and the skin around the wound in the direction resisting the physiological tension applied in the dehiscence direction. In addition, this makes it possible to consistently reduce the physiological tension acting on the skin around the sutured wound in the dehiscence direction and the tension acting on the suture thread associated with the physiological tension by slightly loosening the skin around the sutured wound from the direction resisting the dehiscence direction to the sutured wound. Therefore, it is possible to incorporate the means for consistently reducing the physiological tension acting on the skin around the sutured wound or the suture thread into the manufacturing process as a function of the wound treatment medical tape and cover and apply the medical tape onto the sutured wound and the skin around the wound as a surface by providing a function of reducing the physiological tension on the entire surface. Therefore, it is possible to efficiently reduce the physiological tension. In addition, it is possible to prevent wound dehiscence by slightly loosening the skin around the sutured wound from the direction resisting the dehiscence direction to the sutured wound and consistently reducing the physiological tension acting on the skin around the sutured wound in the dehiscence direction and the tension acting on the suture thread associated with the physiological tension by virtue of the effective shrink force. The inventor completed the present invention on the basis of such knowledge. The term “slightly loosening the skin around the wound” as used herein does not necessarily mean a visually recognizable level, but also includes an insignificant level recognizable by using a magnification lens or microscope. In addition, the term “tape” as used herein refers to a thin band-shaped tape having a narrow width, but the shape is not limited thereto depending on the use purpose.

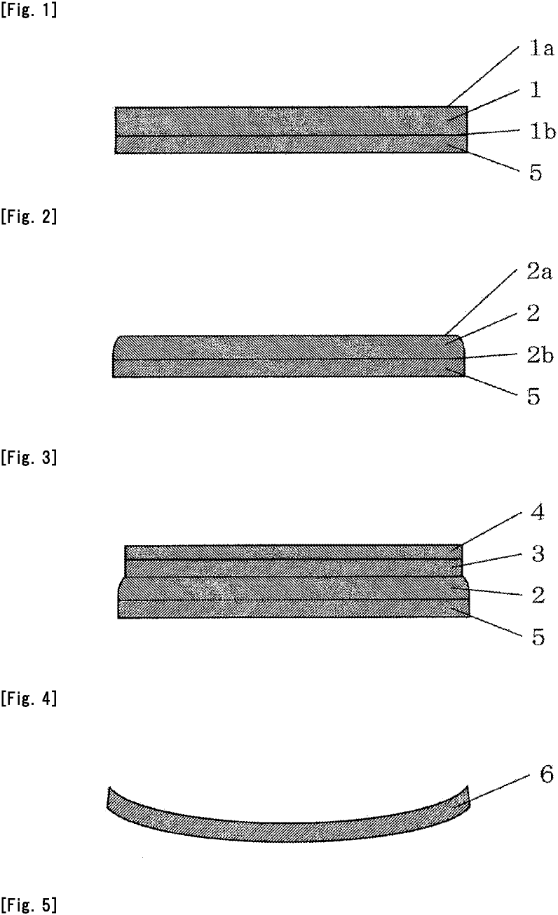

In the wound treatment medical tape obtained by the manufacturing method according to the present invention, a solution polymer for making the elastic base member portion is applied to the upper layer of the internal stress holding portion. In this case, focusing on the cross-sectional shape of the solution polymer, an upper base of the solution polymer not attached to the internal stress holding portion and a lower base of the solution polymer appropriately attached to the internal stress holding portion in a laminated state have the same length and a rectangular shape (FIG. 1). Then, the effective shrink force is held in the elastic base member portion by the internal stress holding portion by completing the film formation of the solution polymer with the effective shrink force being provided in the elastic base member portion, and completing a lamination of the elastic base member portion and the internal stress holding portion. The cross-sectional shape of the elastic base member portion in this case is trapezoidal having a thickness thinner than that of the solution polymer immediately after applying the solution polymer of FIG. 1 and an upper base slightly shorter than the lower base (FIG. 2). This shows that the film formation of the solution polymer is completed with the effective shrink force being provided in the elastic base member portion without freely shrinking the upper base of the solution polymer, a lamination of the elastic base member portion and the internal stress holding portion is completed concurrently, and the effective shrink force is held in the elastic base member portion by the internal stress holding portion. Note that, in the manufacturing process, when the upper base of the solution polymer is freely shrunken without a limitation by virtue of the internal stress associated with the forming shrinkage of the solution polymer, is then cured to complete the film formation to obtain the elastic base member portion, and is laminated with the internal stress holding portion concurrently, the lower base of the elastic base member portion has an internal stress associated with the forming shrinkage of the solution polymer. However, the upper base of the elastic base member portion does not have the internal stress associated with the forming shrinkage of the solution polymer, and only the lower base of the elastic base member portion is shrunken even when the internal stress holding portion is removed. Therefore, the entire elastic base member portion is not shrunken, and it is difficult to hold the target effective shrink force in the elastic base member portion in the manufacturing process. In addition, the physiological tension acting on the wound and the skin around the wound after suturing does not necessarily have a visually recognizable level, but also includes an insignificant level such as a magnification lens or microscope level. For this reason, the effective shrink force held in the elastic base member portion may be slight, and the effect of the present invention can be sufficiently exhibited even when the effective shrink force is slight.

Examples of the present invention will be described hereinafter in details with reference to the accompanying drawings, but the present invention is not limited thereto. According to the present invention, in a process of appropriately selecting, combining, and manufacturing materials of the wound treatment medical tape, an inelastic plate-shaped plastic film having a function of holding an internal stress associated with forming shrinkage in the elastic base member portion is selected as the internal stress holding portion. A solution polymer (urethane resin liquid and cross-linking agent liquid) as a material of a polyurethane film for making the elastic base member portion is applied to the upper layer of the internal stress holding portion (FIG. 1). Then, the film formation of the solution polymer is completed, and a lamination of the elastic base member portion and the internal stress holding portion is completed concurrently with the effective shrink force being provided in the elastic base member portion, so that the effective shrink force in the elastic base member portion is held by the internal stress holding portion (FIG. 2). Then, an acryl-based adhesive as the adhesion portion having a function of applying and holding the elastic base member portion to skin is applied on a surface of the elastic base member portion opposite to the internal stress holding portion. Then, a release sheet having a release agent coated on a surface of high-quality paper is provided as a release portion having a function of protecting the adhesion portion, so that the wound treatment medical tape is obtained (FIG. 3). Note that this wound treatment medical tape is based on a use method including covering a wound and skin around the wound with the adhesion portion to apply the tape to the skin, and removing the internal stress holding portion to release the effective shrink force held in the elastic base member portion to a direction resisting the physiological tension so as to reduce a physiological tension acting in the dehiscence direction by the internal stress holding portion. According to the present invention, in the manufacturing process of the wound treatment medical tape, it is essential to hold the effective shrink force in the elastic base member portion.

According to the first embodiment of the present invention as illustrated in FIGS. 1 to 3, the wound treatment medical tape has a solution polymer (urethane resin liquid and cross-linking agent liquid) 1 as a source material of the elastic base member portion 2, an elastic base member portion 2 that is film-formed of the solution polymer (urethane resin liquid and cross-linking agent liquid) 1 as a source material of the elastic base member portion 2, has an elastic function, and covers a wound and skin around the wound, an adhesion portion 3 having a function of applying and holding the elastic base member portion 2 to the skin, a release portion 4 having a function of protecting the adhesion portion 3, and an inelastic internal stress holding portion 5 having a function of holding the effective shrink force in the elastic base member portion 2. The wound treatment medical tape includes the internal stress holding portion 5, the elastic base member portion 2, the adhesion portion 3, and the release portion 4 sequentially from the bottom.

FIG. 1 is a diagram illustrating a manufacturing process for the wound treatment medical tape according to the first embodiment of the present invention, and is a schematic cross-sectional view illustrating a laminating process in which the solution polymer 1 for making a material of the elastic base member portion 2 is applied to the upper layer of the internal stress holding portion 5. In the wound treatment medical tape according to the first embodiment, an inelastic plastic film formed in a flat shape to provide a function of holding the effective shrink force in the elastic base member portion 2 is selected as the internal stress holding portion 5, and the solution polymer (urethane resin liquid and cross-linking agent liquid) 1 as a source material of the polyurethane film for making the elastic base member portion 2 is applied to the upper layer of the internal stress holding portion 5. In this case, the cross-sectional shape of the solution polymer includes an upper base 1a of the solution polymer (not attached to the internal stress holding portion) and a lower base 1b of the solution polymer (appropriately attached to the internal stress holding portion in the laminated state) having the same length and the rectangular shape as shown in FIG. 1. Note that reference numeral 1 refers to the solution polymer, reference numeral 1a refers to the upper base of the solution polymer, reference numeral 1 b refers to the lower base of the solution polymer, and reference numeral 5 refers to the internal stress holding portion.

FIG. 2 is a schematic cross-sectional view illustrating a wound treatment medical tape manufacturing process according to the first embodiment of the present invention, in which the film formation of the solution polymer 1 is completed while the effective shrink force is provided in the elastic base member portion 2 by rapidly curing the solution polymer 1 from the state that the solution polymer 1 for making the elastic base member portion 2 is applied to the upper layer of the internal stress holding portion 5 as shown in FIG. 1, a lamination of the elastic base member portion 2 and the internal stress holding portion 5 is completed concurrently so as to allow the internal stress holding portion 5 to hold the effective shrink force in the elastic base member portion 2. Referring to the cross-sectional shape of the solution polymer 1 of FIG. 1, the upper base 1a of the solution polymer (not attached to the internal stress holding portion) and the lower base 1b of the solution polymer (appropriately attached to the internal stress holding portion 5 in the laminated state) have the same length and the rectangular shape as shown in FIG. 1. Then, as shown in FIG. 2, in order to complete the film formation of the solution polymer 1 with the effective shrink force being provided in the elastic base member portion 2, the solution polymer 1 is rapidly cured and film-formed to obtain the elastic base member portion 2, and a lamination of the elastic base member portion 2 and the internal stress holding portion 5 is completed concurrently so as to allow the internal stress holding portion 5 to hold the effective shrink force in the elastic base member portion 2. As a result, shrinkage caused by the internal stress associated with forming shrinkage of the solution polymer 1 does not occur in the lower base 2b of the elastic base member portion appropriately attached to the internal stress holding portion 5 in the laminated state concurrently with the film formation of the elastic base member portion 2. Meanwhile, since the upper base 2a of the elastic base member portion is not attached to the internal stress holding portion 5, it is slightly shrunken due to the internal stress associated with forming shrinkage of the solution polymer 1. However, since the solution polymer 1 is rapidly cured and film-formed in order to complete the film formation of the solution polymer 1 with the effective shrink force being provided in the elastic base member portion 2, the upper base 1a of the solution polymer is not freely shrunken, and the cross-sectional shape of the elastic base member portion 2 is trapezoidal such that the upper base 2a of the elastic base member portion is slightly shorter than the lower base 2b of the elastic base member portion. This shows that the film formation of the solution polymer 1 is completed with the effective shrink force being provided in the elastic base member portion 2, and a lamination of the elastic base member portion 2 and the internal stress holding portion 5 is completed concurrently. In addition, this also shows that the internal stress holding portion 5 can hold the effective shrink force in the elastic base member portion 2. Note that reference numeral 2 refers to the elastic base member portion, reference numeral 2a refers to an upper base of the elastic base member portion, reference numeral 2b refers to a lower base of the elastic base member portion, and reference numeral 5 refers to the internal stress holding portion.

FIG. 3 is a cross-sectional view illustrating a medical tape manufacturing process according to the first embodiment of the present invention. An adhesive as an adhesion portion 3 having a function of applying and holding the elastic base member portion 2 to skin is applied to the upper layer of the elastic base member portion 2 of FIG. 2, and a release sheet as a release portion 4 having a function of protecting the adhesion portion 3 is provided on the upper layer of the adhesion portion 3, so that the wound treatment medical tape is obtained. Note that reference numeral 2 refers to the elastic base member portion, reference numeral 3 refers to the adhesion portion, reference numeral 4 refers to the release portion, and reference numeral 5 refers to the internal stress holding portion.

In the manufacturing process of the wound treatment medical tape, as shown in FIG. 2, the film formation of the solution polymer is completed with the effective shrink force being provided in the elastic base member portion, and a lamination of the elastic base member portion and the internal stress holding portion is completed concurrently, so as to obtain the elastic base member portion that holds the effective shrink force in the elastic base member portion by the internal stress holding portion. The procedure of using the wound treatment medical tape includes: (1) remove the release portion; (2) apply the adhesion portion to a wound and skin around the wound; and (3) remove the internal stress holding portion. When the internal stress holding portion is removed on the basis of this use procedure, the effective shrink force held in the elastic base member portion is released to the wound and skin around the wound to which the adhesion portion is applied. The released effective shrink force acts in the direction resisting the physiological tension applied in the dehiscence direction so as to shrink the elastic base member portion in the direction resisting the physiological tension , shrink the wound and the skin around the wound in the direction resisting the physiological tension acting in the dehiscence direction, and slightly loosen the skin around the sutured wound from the direction resisting the dehiscence direction. Therefore, it is possible to consistently reduce the physiological tension acting on the skin around the sutured wound in the dehiscence direction and the tension acting on the suture thread associated with the physiological tension. In addition, the medical tape has a function of reducing the physiological tension on the entire surface of the tape, and can be applied by covering the skin as a surface. Therefore, since the released effective shrink force acts on the skin around the medical tape application portion as well as the wound and the skin around the wound covered by the medical tape, it becomes a force of attracting the skin around the wound by the effective shrink force. As a result, the wound and its surrounding tissues can be stably maintained, and the physiological tension acting on the wound and the skin around the wound in the dehiscence direction can be reduced. Therefore, it is possible to provide a high effect of preventing the wound dehiscence associated with the physiological tension and provide a method of manufacturing a wound treatment medical tape easy to use and apply regardless of whether the wound is sutured or not.

Specifically, according to the first embodiment of the present invention, the urethane resin liquid and the cross-linking agent liquid are selected as the solution polymer 1, the polyurethane film formed of the solution polymer 1 to cover the wound and the skin around the wound and provide an elastic function is selected as the elastic base member portion 2, an acrylic adhesive having a function of applying and holding the elastic base member portion 2 to the skin is selected as the adhesion portion 3, a release sheet obtained by coating a release agent on a surface of high-quality paper having a function of protecting the adhesion portion 3 is selected as the release portion 4, and an inelastic plate-shaped plastic film having a function of holding the internal stress associated with forming shrinkage in the elastic base member portion 2 is selected as the internal stress holding portion 5. In the manufacturing process, the urethane resin liquid and the cross-linking agent liquid as the solution polymer 1 are applied to the upper layer of the inelastic plate-shaped plastic film as the internal stress holding portion 5. Then, in order to complete the film formation of the solution polymer 1 with the effective shrink force being provided in the elastic base member portion 2, the film formation is performed by rapidly curing the solution polymer 1 so that the elastic base member portion 2 is obtained. Concurrently, a lamination of the elastic base member portion 2 and the internal stress holding portion 5 is completed, so that the effective shrink force is held in the elastic base member portion 2 by the internal stress holding portion 5. As a result, the upper base 1a of the solution polymer is not attached to the internal stress holding portion 5, so that it is slightly shrunken as the solution polymer 1 is cured. The lower base 1b of the solution polymer appropriately attached while being laminated with the internal stress holding portion 5 concurrently with the film formation of the elastic base member portion 2 is not shrunken along with the curing. In addition, the cross-sectional shape of the elastic base member portion 2 after the film formation is trapezoidal such that the upper base 2a of the elastic base member portion is slightly shorter than the lower base 2b of the elastic base member portion. This shows that the upper base 1a of the solution polymer is not freely shrunken by rapidly curing and a film formation of the solution polymer 1, the film formation of the solution polymer 1 is completed with the effective shrink force being provided in the elastic base member portion 2, and a lamination of the elastic base member portion 2 and the internal stress holding portion 5 is completed concurrently, so that the effective shrink force can be held in the elastic base member portion 2 by the internal stress holding portion 5. Then, the acrylic adhesive as the adhesion portion 3 and the release sheet obtained by coating a release agent on a surface of high-quality paper as the release portion 4 are provided sequentially on the surface of the elastic base member portion 2 opposite to the internal stress holding portion 5.

The urethane resin liquid and the cross-linking agent liquid are used as a material of the solution polymer in the first embodiment. Alternatively, any solution polymer material or other new materials may be employed, or another solution polymer material may be mixed or laminated, as long as the effective shrink force can be held in the elastic base member portion by the internal stress holding portion by applying it to the upper layer of the internal stress holding portion, completing the film formation of the solution polymer with the effective shrink force being provided in the elastic base member portion, and concurrently completing a lamination of the elastic base member portion and the internal stress holding portion. In addition, an inelastic plate-shaped plastic film formed in the flat shape having a function of holding the effective shrink force in the elastic base member portion is employed as a material of the internal stress holding portion. Alternatively, a cut may be inserted into the plate-shaped plastic film depending on application or convenience, a band-shaped tape covering the cut may be employed, or any material or shape may be employed without a limitation as long as it has a function of holding the effective shrink force in the elastic base member portion. In addition, an acrylic adhesive is employed as a material of the adhesion portion. Alternatively, any adhesive may be employed as long as it has a function of applying and holding the elastic base member portion to the skin and is usable for medical purposes. In addition, a portion to which the adhesive is applied may be a part or the entire surface depending on application, and its material, shape, or the like is not limited. Furthermore, while a release sheet formed by coating a release agent on a surface of high-quality paper is employed as a material of the release portion, any material may be employed as long as it has a function of protecting the adhesion portion. For example, the release portion may be formed by coating a release agent on a surface of the elastic base member portion opposite to the internal stress holding portion, a package such as a wrapping sheet may be employed as the release portion, and its material, shape, and the like are not limited. Note that the materials of each part of the wound treatment medical tape, a combination of the materials, functions to be added, and the like may be arbitrary depending on the application, design, convenience, or the like, without any limitation as long as the effects of the present invention can be achieved.

Next, a method of manufacturing the wound treatment medical tape according to the second embodiment of the present invention will be described with reference to FIG. 4. In the method of manufacturing the wound treatment medical tape according to the first embodiment of the present invention, a plate-shaped plastic film formed in a flat shape is used as the internal stress holding portion 5 to set the shrink direction of the effective shrink force to a centripetal direction (omnidirectional). However, according to the second embodiment of the present invention, in order to manufacture the wound treatment medical tape by limiting the shrink direction of the effective shrink force to one direction (vertical direction), in the manufacturing process, the horizontal shrink force of the effective shrink force existing in the elastic base member portion after the film formation is eliminated or reduced. In order to efficiently exert the vertical effective shrink force when the wound treatment medical tape is applied, the internal stress holding portion 6 is shaped as shown in FIG. 4. In the internal stress holding portion 6, the inelastic plastic film having flexibility and a restoring function corresponding to a shape change is formed in a substantially arcuate shape having a recessed cross-sectional shape in the horizontal direction. In addition, although not shown in the drawings, in the manufacturing process, the cross-sectional shape of the internal stress holding portion 6 formed in a substantially arcuate shape is maintained to have a flat shape by an external force generated by a manufacturing facility or the like. The solution polymer is applied to the upper layer of the internal stress holding portion 6, the film formation of the solution polymer is completed with the effective shrink force being provided in the elastic base member portion, and a lamination of the elastic base member portion and the internal stress holding portion 6 formed in a substantially arcuate shape is completed, so that the effective shrink force is held in the elastic base member portion by the internal stress holding portion 6 formed in a substantially arcuate shape. Then, as the external force generated by the manufacturing facility or the like is removed, the horizontal cross-sectional shape is restored to the substantially arcuate shape, which is a recessed shape, by virtue of a restoring function provided in the internal stress holding portion 6 formed in a substantially arcuate shape. As a result, it is possible to obtain the elastic base member portion in which the horizontal effective shrink force in the elastic base member portion is eliminated or reduced. Then, through a process of laminating the adhesion portion having a function of applying and holding the elastic base member portion to skin and a release portion having a function of protecting the adhesion portion on a surface of the elastic base member portion, in which the horizontal effective shrink force in the elastic base member portion is eliminated, opposite to the internal stress holding portion 6 formed in a substantially arcuate shape, a wound treatment medical tape is obtained by limiting the shrink direction of the effective shrink force to one direction. Note that, in the second embodiment of the present invention, in order to obtain the elastic base member portion in which the horizontal effective shrink force in the elastic base member portion is eliminated or reduced, in the manufacturing process, the shape of the internal stress holding portion 6 is set to the substantially arcuate shape. However, in the manufacturing process described above, any shape may be employed without a limitation as long as it can unidirectionally eliminate or reduce the effective shrink force in the elastic base member portion. In addition, in the manufacturing process, the cross-sectional shape of the internal stress holding portion 6 formed in a substantially arcuate shape is maintained to have the flat shape by using the external force from a manufacturing facility or the like. Alternatively, it may have any shape as long as the horizontal effective shrink force in the elastic base member portion can be eliminated or reduced. In this case, maintaining the cross-sectional shape to have the flat shape is not essential. In addition, in the manufacturing process, for adjusting the reduction degree of the horizontal effective shrink force in the elastic base member portion, both ends of the internal stress holding portion 6 formed in a substantially arcuate shape may be cut out after removing the external force applied from the manufacturing facility or the like to the internal stress holding portion 6 formed in a substantially arcuate shape. Furthermore, the reduction degree of the horizontal effective shrink force in the elastic base member portion may be arbitrary depending on the application, manufacturing cost, and the like.

All of the medical tapes obtained through the manufacturing methods according to the present invention as shown in FIGS. 1 to 4 have a basic structure of the medical tape obtained through the manufacturing method according to the present invention. In such a configuration, it is possible to incorporate a means for consistently reducing the physiological tension acting on a sutured wound and skin around the wound in the dehiscence direction into the wound treatment medical tape as a function. In the manufacturing process of this wound treatment medical tape, it is possible to manufacture a medical tape capable of holding the effective shrink force in the elastic base member portion and apply it to skin with the effective shrink force being held in the elastic base member portion at the time of application of this medical tape. In addition, in this wound treatment medical tape, the effective shrink force held in the elastic base member portion can be released to the wound and the skin around the wound, to which the adhesion portion is applied, by removing the internal stress holding portion after applying the adhesion portion to the skin. In this case, the elastic base member portion is shrunken by the released effective shrink force in the direction resisting the physiological tension so as to shrink the wound and the skin around the wound in the direction resisting the physiological tension acting in the dehiscence direction and slightly loosen the skin around the sutured wound from the direction resisting the dehiscence direction to the sutured wound. In addition, the physiological tension acting on the skin around the sutured wound in the dehiscence direction and the tension acting on the suture thread associated with the physiological tension are consistently reduced. Therefore, it is possible to provide a wound treatment medical tape having a high effect of preventing the wound dehiscence and easy to use and apply regardless of whether the wound is sutured or not. Note that, although the shrink force of the wound treatment medical tape is expressed as “slightly loosening the skin around the wound” herein, this is not necessarily a visually recognizable level and also includes an insignificant state such as a level recognizable by using a magnification lens or microscope. This is because the physiological tension acting on the skin around the sutured wound in the dehiscence direction and the tension acting on the suture thread associated with the physiological tension are not necessarily a visually recognizable level but also an insignificant level such as a level recognizable by using a magnification lens or microscope in some cases. Therefore, the effective shrink force held in the elastic base member portion may be insignificant. The effects of the present invention can be sufficiently achieved even when it is insignificant.

In the first embodiment described hereinbefore, the wound treatment medical tape has a rectangular shape. However, the shape of the medical tape may be selected freely depending on the application. For example, the medical tape may have a roll shape or sheet shape and may be appropriately shaped depending on each part of a patient’s body. In addition, this medical tape basically has the internal stress holding portion, the elastic base member portion, the adhesion portion, and the release portion from the bottom. However, a gauze, pad, or the like may be provided on the upper layer of the adhesion portion depending on the application, design, convenience, and the like. Note that, although the medical tape according to the present invention is basically closely applied to skin, the medical tape may be applied by inserting a protective material for protecting skin from a medical adhesive or the like in some cases.

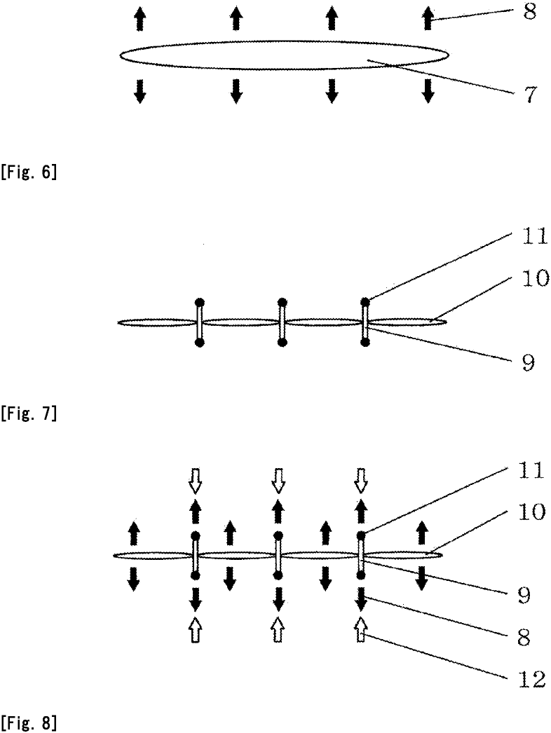

Next, the physiological tension or the wound dehiscence associated with the suture thread used to suture a wound and a use method or effect of the wound treatment medical tape according to the second embodiment of the present invention will be described with reference to FIG. 5 to FIG. 10. FIG. 5 is a schematic plan view illustrating a state of the wound surface after incising the skin in a straight line shape. Note that reference numeral 7 refers to a wound surface, reference numeral 8 refers to the physiological tension, and the arrows refer to directions thereof. When the skin is incised in a straight line shape with a surgical scalpel, the shape of the wound surface 7 is not limited to the straight line shape. As shown in FIG. 5, the physiological tension 8 acting on the skin around the wound in the dehiscence direction makes the shape from the straight line shape to an oval shape. FIG. 6 is a schematic plan view illustrating a state that the skin around the wound of FIG. 5 is sutured with the suture thread. Note that reference numeral 9 refers to a suture thread, reference numeral 10 refers to a sutured wound surface, and reference numeral 11 refers to a portion through which the suture thread penetrates. When suturing the wound surface 7 of FIG. 5, it is necessary to attract the wound surface 7 in an oval open state with a suture thread or the practitioner’s fingers such that the incised wound surface 7 has a straight line shape. In this case, since the prognosis is better when the spacing between the suture threads for suturing the skin around the wound is coarse rather than dense, the spacing of the suture threads becomes inevitably coarse, and the state of the suture thread is moderately loosened as shown in FIG. 6 in order to prevent overtightening, which results in poor prognosis.

FIG. 7 is a schematic plan view illustrating a force applied to a peripheral portion (wound edge) of the sutured wound of FIG. 6. Note that reference numeral 8 refers to the physiological tension in which the arrows refer to directions, and the lengths refer to the magnitudes, reference numeral 9 refers to the suture thread, reference numeral 10 refers to a sutured wound surface, reference numeral 11 refers to a portion through which the suture thread penetrates, reference numeral 12 refers to a force for attracting the wound surface by the suture thread, in which the arrows refer to directions, and the lengths refer to the magnitudes. In the suturing of the wound surface 7 of FIG. 5, the skin around the wound is attracted and sutured with a suture thread or the practitioner’s fingers such that the shape of the wound surface 7 to be sutured is changed from the open state to the closed state, and the suture thread is tied. Therefore, at the wound edge around the wound, as shown in FIG. 7, a force 12 of closing the wound surface by the suture thread and a force of opening the wound surface by the physiological tension 8 act in an antagonistic state, and the suture thread and the skin around the wound are attractingly combined with each other at all times. Furthermore, at the portion 11 through which the suture thread penetrates, the wound surface is maintained in a closed state because the force 12 of attracting the wound surface by the suture thread and the force of opening the wound surface by the physiological tension 8 work in an antagonistic state. However, even on the sutured wound surface 10, the spacing between the suture threads is coarse, and at a part of the wound surface between the suture threads that the force 12 of attracting the wound surface by the suture thread does not reach, the force of opening the wound surface by the physiological tension 8 is stronger, so that the shape of the wound surface between the suture threads becomes a small oval shape. Furthermore, in a part of the skin around the portion 11 through which the suture thread penetrates, the force 12 of attracting the wound surface by the suture thread and the force of opening the wound surface by the physiological tension 8 act in an antagonistic manner, so that the tension caused by the physiological tension 8 is concentrated on the wound surface side of the portion 11 through which the suture thread penetrates, that is, the skin tissue inward of the suture thread. When consistent compression by the suture thread is applied to the skin tissue on the wound surface side, poor blood circulation occurs in the skin tissue on the wound surface side, the skin tissue falls into compression necrosis, and this compression necrosis gradually advances to the skin tissue on the wound surface side, tears the skin around the wound, causes the suture thread to be removed, and causes wound dehiscence in some cases. Note that, under the current situation, even re-suturing is not expected to be effective for the wound dehiscence mainly caused by the necrosis of the skin around the wound or the like (medical tool compression wound caused by the suture thread).

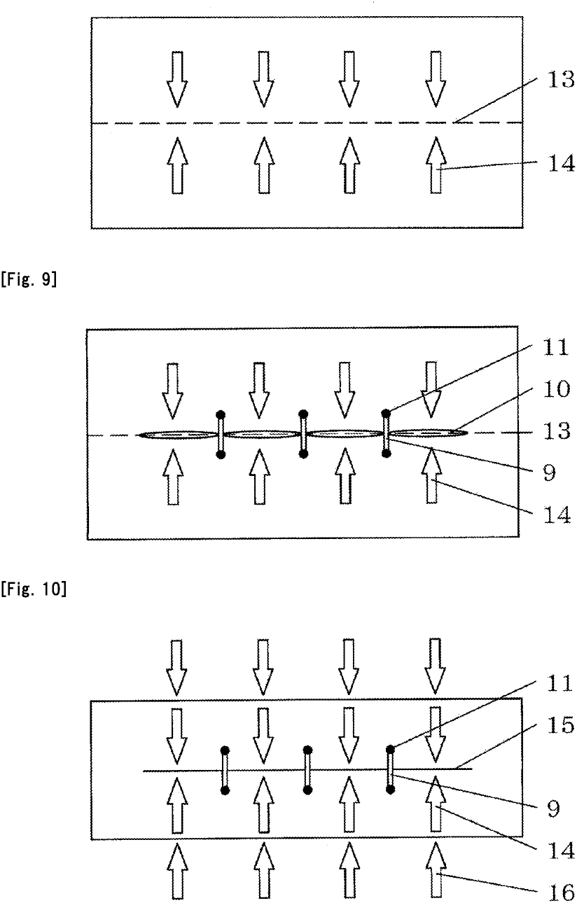

FIG. 8 is a schematic plan view illustrating a medical tape according to the second embodiment of the present invention. The medical tape is a wound treatment medical tape in which the shrink direction of the effective shrink force is limited to the vertical direction. Note that reference numeral 13 refers to a broken line provided in the center of the medical tape, and reference numeral 14 refers to the directions and the magnitudes of the effective shrink force. The broken line 13 provided in the center of the medical tape is printed on the internal stress holding portion of the medical tape, and is for facilitating the procedure of overlappingly applying the tape to the wound at the time of use. The effective shrink force 14 is held in the elastic base member portion by the function of the internal stress holding portion. FIG. 9 is a schematic plan view illustrating a medical tape according to the second embodiment of the present invention used for the sutured wound of FIG. 6. Note that reference numeral 9 refers to the suture thread, reference numeral 10 refers to the sutured wound surface, reference numeral 11 refers to a portion through which the suture thread penetrates, reference numeral 13 refers to a broken line provided in the center of the medical tape, and reference numeral 14 refers to directions and magnitudes of the effective shrink force. First, a medical tape having a size capable of appropriately covering the sutured wound and its surrounding skin is selected. Then, the release portion is removed from the medical tape, and the sutured wound and its surrounding skin are covered by the elastic base member portion such that the broken line 13 provided in the center of the medical tape and the sutured wound surface 10 of FIG. 6 overlap each other, and the medical tape is applied to the skin by the adhesion portion. At this time, since the internal stress holding portion is not removed, the effective shrink force 14 is held in the elastic base member portion by virtue of the function of the internal stress holding portion.

FIG. 10 is a schematic cross-sectional view illustrating the effect of using the medical tape according to the second embodiment of the present invention. Note that reference numeral 9 refers to the suture thread, reference numeral 11 refers to a portion through which the suture thread penetrates, reference numeral 14 refers to directions and magnitudes of the effective shrink force, reference numeral 15 refers to a wound surface closed by the effective shrink force, and reference numeral 16 refers to a force of attracting the skin around the wound by the effective shrink force. FIG. 10 shows a state that the effective shrink force 14 held in the elastic base member portion and the force 16 of attracting the skin around the wound by the effective shrink force are exerted by removing the internal stress holding portion on which the broken line 13 provided in the center of the medical tape is printed, from the state of FIG. 9. In this medical tape, since the shrink direction of the effective shrink force is limited to the vertical direction, it is possible to efficiently exert the vertical effective shrink force 14 and consistently reduce the physiological tension acting on the skin around the sutured wound surface 10 in the dehiscence direction by covering as a surface the skin around the sutured wound surface 10 in FIG. 9 to apply the medical tape to the skin, and removing the internal stress holding portion. Therefore, when the wound is sutured with the suture thread, the shape of the sutured wound surface 10 of FIG. 7, that is, the shape of the wound surface between the suture threads is a small oval shape because the force of opening the wound surface by the physiological tension 8 in FIG. 7 is stronger on the wound surface between the suture threads where the force 12 of attracting the wound surface by the suture thread of FIG. 7 does not reach. By using this medical tape, referring to FIG. 10, the effective shrink force 14 corresponding to reduction of the vertical length from the medical tape of FIG. 9 is exerted, so that the wound surface 15 is closed by the effective shrink force. In addition, the medical tape has a function of reducing the physiological tension on the entire surface of the tape, and can be applied by covering the skin as a surface. Therefore, the released effective shrink force 14 acts not only on the skin around the sutured wound surface 10 covered by the medical tape but also on the skin around the medical tape application portion, so that a force 16 of attracting the skin around the wound is generated by the effective shrink force. As a result, it is possible to reduce the tension load caused by the physiological tension 8 concentrated on the wound surface side of the portion 11 through which the suture thread penetrates, that is, on the skin tissue inward of the suture thread. It is possible to avoid a virtuous circle, that is, “when consistent compression by the suture thread is applied to the skin tissue on the wound surface side, poor blood circulation occurs in the skin tissue on the wound surface side, the skin tissue falls into compression necrosis, and this compression necrosis gradually advances to the skin tissue on the wound surface side, tears the skin around the wound, causes the suture thread to be removed, and causes wound dehiscence in some cases”. Therefore, it becomes realistic to prevent the wound dehiscence mainly caused by necrosis of the skin around the wound (medical tool compression wound caused by suture thread). Furthermore, even for the dermis suturing described in paragraph 0003, the medical tape according to the present invention becomes an effective means for minimizing scars on the skin surface, and it may be possible to eliminate the necessity of epidermal suturing and also avoid needle marks on the skin surface.

According to the manufacturing method of the wound treatment medical tape of the present invention, the effective shrink force is held in the elastic base member portion during the manufacturing process, and at the time of use, the medical tape covers the sutured wound and the skin around the wound as a surface with the effective shrink force being held in the elastic base member portion, and is applied to the skin with the adhesion portion, and the internal stress holding portion is removed. As a result, the effective shrink force in the elastic base member portion is released, so as to allow the effective shrink force to efficiently act in the direction resisting the physiological tension applied in the dehiscence direction, reduce the physiological tension acting on the skin around the sutured wound in the dehiscence direction, and provide a high effect in wound dehiscence prevention. Therefore, it is possible to provide a method of manufacturing the wound treatment medical tape easy to use and apply regardless of whether or not the wound is sutured, and the present invention greatly contributes to the medical industry.

REFERENCE SIGNS LIST

| | |
|---|---|
| 1 | solution polymer |
| 1 a | solution polymer upper base |
| 1 b | solution polymer lower base |
| 2 | elastic base member portion |
| 2 a | elastic base member portion upper base |
| 2 b | elastic base member portion lower base |
| 3 | adhesion portion |
| 4 | release portion |
| 5 | internal stress holding portion |
| 6 | internal stress holding portion formed in substantially arcuate shape according to the second embodiment of the present invention |
| 7 | wound surface |
| 8 | physiological tension |
| 9 | suture thread |
| 10 | sutured wound surface |
| 11 | portion through which suture thread penetrates |
| 12 | force of attracting wound surface by suture thread |
| 13 | broken line in center of medical tape according to the second embodiment of the present invention |
| 14 | effective shrink force |
| 15 | wound surface closed by effective shrink force |
| 16 | force of attracting skin around the wound by effective shrink force |

1. A method of manufacturing a wound treatment medical tape, the wound treatment medical tape comprising
an elastic base member portion that covers a wound and a skin around the wound and has an elastic function, an adhesion portion having a function of applying and holding the elastic base member portion to the skin, a release portion having a function of protecting the adhesion portion, and an internal stress holding portion having a function of holding an effective shrink force in the elastic base member portion, wherein
the internal stress holding portion, the elastic base member portion, the adhesion portion, and the release portion are provided in this order from a bottom layer,
the method comprises:
a step of applying a solution polymer for making the elastic base member portion to an upper layer of the internal stress holding portion;
a step of subsequently holding the effective shrink force in the elastic base member portion with the internal stress holding portion by completing a film formation of the solution polymer with the effective shrink force being held in the elastic base member portion and concurrently completing a lamination of the elastic base member portion and the internal stress holding portion; and
a step of subsequent laminating the adhesion portion and the release portion on a surface of the elastic base member portion opposite to the internal stress holding portion, and
the method releases the effective shrink force existing in the elastic base member portion by removing the internal stress holding portion at a time of use.
